(19) United States
(12) Patent Application Publication
(10) Pub. No.: US 2023/0197231 A1
(43) Pub. Date: Jun. 22, 2023

(54) PERSONALIZED DETERMINATION OF DRUG CONTRAINDICATIONS USING BIOCHEMICAL KNOWLEDGE GRAPHS

(71) Applicant: United Health Group Incorporated, Minnetonka, MN (US)

(72) Inventors: Irfan Bulu, Sartell, MN (US); Cem Unsal, Alameda, CA (US)

(21) Appl. No.: 18/082,868

(22) Filed: Dec. 16, 2022

Related U.S. Application Data
(60) Provisional application No. 63/265,579, filed on Dec. 17, 2021.

(51) Int. Cl. G16H 20/10

(52) U.S. Cl. CPC G16H 20/10

(57) ABSTRACT

Various embodiments of the present disclosure disclose generating contraindication alert communications. A knowledge graph data structure, including a graph-based representation associated with a user identifier and having nodes and edges, is accessed. Edge weights are adjusted based on medical data associated with the user identifier. One or more sequential traversals of the knowledge graph data structure are performed until an equilibrium condition is met. Based on determining that a subset of nodes is associated with visit tallies totaling more than a threshold proportion of all node visits associated with the one or more sequential traversals, a contraindication alert communication, which includes representation of a biological effect for the user identifier, can be generated and transmitted.

### CROSS-REFERENCE TO RELATED APPLICATIONS

This application claims the benefit of U.S. Provisional Application No. 63/265,579, entitled “PERSONALIZED DETERMINATION OF DRUG CONTRAINDICATIONS USING BIOCHEMICAL KNOWLEDGE GRAPHS,” and filed Dec. 17, 2021, the entire contents of which are hereby incorporated by reference.

### BACKGROUND

Various embodiments of the present disclosure address technical challenges related to identification of drug contraindications specific to an individual patient. Various embodiments of the present disclosure make important contributions to various existing drug contraindication identification approaches by addressing each of these technical challenges.

### BRIEF SUMMARY

Various embodiments of the present disclosure disclose generating contraindication alert communications. A knowledge graph data structure, including a graph-based representation associated with a user identifier and having nodes and edges, is accessed. Edge weights are adjusted based on medical data associated with the user identifier. Sequential traversals of the knowledge graph data structure are performed until an equilibrium condition is met. Based on determining that a subset of nodes is associated with visit tallies totaling more than a threshold proportion of all node visits associated with the one or more sequential traversals, a contraindication alert communication, which includes a representation of a biological effect for the user identifier, may be generated and transmitted.

### BRIEF DESCRIPTION OF THE SEVERAL VIEWS OF THE DRAWINGS

FIG. 1 is a schematic diagram showing an example computing system for generating predictive insights using distributions of non-descriptive identifiers in accordance with one or more embodiments of the present disclosure.

FIG. 2 is a schematic diagram showing an example system computing architecture in accordance with some embodiments discussed herein.

FIG. 3 depicts an example knowledge graph data structure in accordance with some embodiments discussed herein.

FIG. 4 is a flowchart showing an example of a process for generating contraindication communications in accordance with some embodiments discussed herein.

FIG. 5 is a flowchart showing an example of a process for traversing a knowledge graph data structure in accordance with some embodiments discussed herein.

FIG. 6 is an operational example of a knowledge graph data structure traversal in accordance with some embodiments discussed herein.

FIG. 7 is an operational example of a knowledge graph data structure traversal in accordance with some embodiments discussed herein.

### DETAILED DESCRIPTION

Various embodiments of the present disclosure are described more fully hereinafter with reference to the accompanying drawings, in which some, but not all embodiments of the present disclosure are shown. Indeed, the present disclosure may be embodied in many different forms and should not be construed as limited to the embodiments set forth herein; rather, these embodiments are provided so that this present disclosure will satisfy applicable legal requirements. The term “or” is used herein in both the alternative and conjunctive sense, unless otherwise indicated. The terms “illustrative” and “example” are used to be examples with no indication of quality level. Terms such as “computing,” “determining,” “generating,” and/or similar words are used herein interchangeably to refer to the creation, modification, or identification of data. Further, “based on,” “based at least in part on,” “based at least on,” “based upon,” and/or similar words are used herein interchangeably in an openended manner such that they do not necessarily indicate being based only on or based solely on the referenced element or elements unless so indicated. Like numbers refer to like elements throughout. Moreover, while some embodiments of the present disclosure are described with reference to predictive data analysis, one of ordinary skill in the art will recognize that the disclosed concepts may be used to perform other types of data analysis.

### I. Overview and Technical Advantages

Embodiments herein provide technological improvements over existing methodologies for identifying possible contraindications for particular individual patients. Embodiments herein employ individualized graph-based representations of a physiological state of a particular patient and systematically and programmatically traverse the graph-based representation to identify possible contraindications for any possible combination of drug or condition specific to the patient.

Physiological phenomena may be effectively modeled in terms of interactions between proteins and other molecules. A disease that a human observer might recognize as a set of symptoms may be described biochemically as levels, relative levels, or changes in levels of particular proteins or other biomolecules. Similarly, drug effects, side effects, physiological measures (e.g., blood pressure), and symptoms may each be described as a set of molecular concentrations representing a state. A model of proteins and biological processes includes nodes representing proteins and biological processes involving molecules (e.g., DNA methylation), cells (e.g., mitosis), tissues (e.g., muscle atrophy), organ systems (e.g., immune response), and the whole organism (e.g., anatomical structure development). In addition to the nodes, the model includes edges describing the relationships between the nodes. The edges are assigned weights that encode the relative importance of the different node types (e.g., protein, drug, disease, process).

The model has demonstrated that effects of drugs are not limited to those molecules that interact directly with the drugs, but may extend to many subsequently interacting molecules and processes resulting in long causal pathways. The effects of drugs (as well as diseases and genes) are seldom discrete and depend on the context in which they are used (e.g., the biochemical state of the person receiving the drugs).

The current standard of care for determining drug contraindications is relatively simple. When a provider wishes to prescribe a drug to a patient, the provider may look up the drug in a database that lists other drugs and diagnoses that are contraindicated for simultaneous use. However, current practice for determining contraindicated combinations of drugs is based on observation of symptoms and drug levels (e.g., on a heterogeneous population of patients) or on a theoretical understanding of the physiological effects of each drug. As a result, many potentially harmful combinations of drugs are currently missed because current drug interaction modeling does not account for complex biochemical interactions. Additionally, current assessments of drug interactions do little to account for the health condition of the particular patient to which the drug is administered and consider only the most general of health conditions such as a diagnosis (e.g., diabetes), range of values (e.g., blood glucose > 180 mg/dl), or genetic testing.

Embodiments of the present disclosure present innovative traversal techniques associated with a knowledge graph data structure specifically generated for a given user identifier so that treatment selection for the user identifier is personalized according to physiological characteristics of the user identifier. Edge weights of the knowledge graph data structure are adjusted according to specific health data associated with the user identifier so that a biased random traversal of the knowledge graph data structure is directed according to the edge weights. The edge weights represent a significance or importance of a relationship between two given nodes, where each node represents a protein, a biological process, a drug, or a molecule. Traversing the knowledge graph data structure in a biased random manner, directed by proportionality of the edge weights, enables treatment to be directed based on significance of possible effects on the user identifier posed by a treatment or aspects of each node.

Approaching treatment using embodiments herein overcome drawbacks associated with generalized contraindication evaluations that do not take into account specific, highly personalized physiological information about a given user identifier. Embodiments herein eliminate the need for trial-and-error approaches to treatments and provide contraindication communications in an amount of time during which data informing the contraindication communications remains relevant. That is, trial-and-error and brute force approaches to identifying contraindications may require intense computing resources and time such that a result is produced after the underlying data or conditions are no longer present or relevant to the analysis.

### II. Examples of Certain Terms

The terms “data,” “content,” “digital content,” “digital content object,” “signal,” “information,” and similar terms may be used interchangeably to refer to data capable of being transmitted, received, and/or stored in accordance with embodiments of the present disclosure. Thus, use of any such terms should not be taken to limit the spirit and scope of embodiments of the present disclosure. Further, where a computing device is described herein to receive data from another computing device, it will be appreciated that the data may be received directly from another computing device or may be received indirectly via one or more intermediary computing devices, such as, for example, one or more servers, relays, routers, network access points, base stations, hosts, and/or the like, sometimes referred to herein as a “network.” Similarly, where a computing device is described herein to send data to another computing device, it will be appreciated that the data may be transmitted directly to another computing device or may be transmitted indirectly via one or more intermediary computing devices, such as, for example, one or more servers, relays, routers, network access points, base stations, hosts, and/or the like.

The term “computer-readable storage medium” refers to a non-transitory, physical or tangible storage medium (e.g., volatile or non-volatile memory), which may be differentiated from a “computer-readable transmission medium,” which refers to an electromagnetic signal.

Examples of non-transitory computer-readable storage media include a magnetic computer readable medium (e.g., a floppy disk, hard disk, magnetic tape, any other magnetic medium), an optical computer readable medium (e.g., a compact disc read only memory (CD-ROM), a digital versatile disc (DVD), a Blu-Ray disc, or the like), a random access memory (RAM), a programmable read only memory (PROM), an erasable programmable read only memory (EPROM), a FLASH-EPROM, or any other non-transitory medium from which a computer may read. The term computer-readable storage medium is used herein to refer to any computer-readable medium except transmission media. However, it will be appreciated that where embodiments are described to use a computer-readable storage medium, other types of computer-readable storage media may be substituted for or used in addition to the computer-readable storage medium in alternative embodiments.

The term “comprising” means including but not limited to and should be interpreted in the manner it is typically used in the patent context. Use of broader terms such as comprises, includes, and having should be understood to provide support for narrower terms such as consisting of, consisting essentially of, and comprised substantially of.

The terms “illustrative,” “example,” “exemplary” and the like are used herein to mean “serving as an example, instance, or illustration” with no indication of qualitative assessment or quality level. Any implementation described herein as “exemplary” is not necessarily to be construed as preferred or advantageous over other implementations.

The phrases “in one embodiment,” “according to one embodiment,” and the like generally mean that the particular feature, structure, or characteristic following the phrase may be included in at least one embodiment of the present disclosure and may be included in more than one embodiment of the present disclosure (importantly, such phrases do not necessarily refer to the same embodiment).

The terms “about,” “approximately,” or the like, when used with a number, may mean that specific number, or alternatively, a range in proximity to the specific number, as understood by persons of skill in the art field.

If the specification states a component or feature “may,” “can,” “could,” “should,” “would,” “preferably,” “possibly,” “typically,” “optionally,” “for example,” “often,” or “might” (or other such language) be included or have a certain characteristic, that particular component or feature is not required to be included or to have the characteristic. Such component or feature may be optionally included in some embodiments, or it may be excluded.

The term “plurality” refers to two or more items.

The term “set” refers to a collection of one or more items.

The term “or” is used herein in both the alternative and conjunctive sense, unless otherwise indicated.

The term “knowledge graph data structure” refers to a data structure arranged according to a graph-based representation and having nodes and edges. The edges of the knowledge graph data structure connect nodes of the knowledge graph data structure. In some embodiments, the knowledge graph data structure is associated with a user identifier. In some embodiments, each node of the nodes represents or is associated with a biological data structure, and each edge of the edges represents a relationship between two nodes it connects. In some embodiments, a node represents one or more of a drug, protein, and/or a biological process involving molecules (e.g., DNA methylation), cells (e.g., mitosis), tissues (e.g., muscle atrophy), organ systems (e.g., immune response), and/or a whole organism (e.g., anatomical structure development).

The term “user identifier” refers to one or more items of data by which a user (e.g., a patient) is uniquely identified. In some embodiments, a user identifier comprises one or more of ASCII text, encryption keys, identification certificates, a pointer, an IP address, a URL, a MAC address, a memory address, an object signature, a HASH value, or other unique identifier, or combinations thereof.

The term “biological data structure” refers to a data structure representative of a medical formulary for use in a knowledge graph data structure. In some embodiments, a biological data structure includes a plurality of records, each containing data associated with the biological data structure. For example, a biological data structure associated with a prescription of Drug A at a certain dosage may include a drug identifier associated with Drug A and a dosage record indicative of that dosage.

The term “edge weight” refers to one or more items of data, associated with an edge of a knowledge graph data structure, representative of a degree of significance of a relationship between two nodes connected by the edge. In some embodiments, the edge weight encodes a relative importance of the different node types (e.g., protein, drug, disease, process).

The term “drug identifier” refers to one or more items of data by which a drug (e.g., a prescription) is uniquely identified. In some embodiments, a drug identifier comprises one or more of ASCII text, encryption keys, identification certificates, a pointer, an IP address, a URL, a MAC address, a memory address, an object signature, a HASH value, or other unique identifier, or combinations thereof.

The term “medical data” refers to one or more items of data representative of medical conditions, history, or the like associated with a user identifier. In some embodiments, medical data includes clinical one or more of notes data, comorbidities data, current medications data, lab values data, insurance claims data, demographic data, medical images data, sensor data, continuous glucose data, or acceleration data indicative of physical activity. In some embodiments, the medical data is associated with dates or timestamps (e.g., temporal aspects).

The term “equilibrium condition” refers to an instance, after one or more sequential traversals of a knowledge graph data structure, when a variation in a proportion of total visits received by each node caused by an additional traversal falls below a variation threshold. That is, upon completion of an additional traversal, a variation in a proportion of total visits received by each node caused by the additional traversal is compared to a variation threshold. If the variation is below the variation threshold, the equilibrium condition is met.

The term “node of interest” refers to a starting node for a traversal of a knowledge graph data structure. In some embodiments, a starting node may be associated with a biological data structure that is a candidate for administering to the user identifier associated with the knowledge graph data structure.

The term “variation threshold” refers to one or more items of data representative of a maximum amount of variation in a proportion of total visits received by each node caused by an additional traversal that is required to identify the presence or absence of an undesirable effect for a given biological data structure. For example, a given biological data structure may be associated with a drug identifier and dosage record, and the given biological data structure may be associated with a variation threshold indicative of whether an undesirable effect is associated with the drug identifier and dosage record.

The term “visit” refers to an instance when a node of a knowledge graph data structure is subject to a traversal. For example, when a system traverses a knowledge graph data structure beginning at a first node, node A, and traverses from node A to node B, via an edge (e.g., edge AB) directly linking node A and node B, both node A and node B are said to have been visited and each is associated with a visit count or tally of at least one (1).

The term “visit tally” refers to a count (e.g., a number) of visits associated with a given traversal of a knowledge graph data structure. There may be a total visit tally (e.g., a total number of all node visits during a given traversal) and there may be individual visit tallies (e.g., each node having its respective number of visits during the given traversal).

The term “biological effect” refers to a possibility of a contraindication associated with prescribing a particular formula (e.g., prescription/dosage) for a particular patient, or a possibility of a positive biological effect (e.g., improvement associated with a biological condition). A contraindication may be a drug-drug contraindication or a drug-condition contraindication. A drug-drug contraindication cautions against using one drug or treatment in combination with another where either alone might be acceptable. A drug-condition contraindication cautions against the use of a drug or treatment with any patient exhibiting particular characteristics, such as a health condition or allergies to the drug.

The term “contraindication communication” refers to an electronic communication configured for rendering according to a receiving computing entity and including instructions for depicting visual representation of one or more of a presence of a contraindication (e.g., undesirable biological effect) or lack of a contraindication (e.g., desirable or improved biological effect), among other items of data associated with a user identifier, biological data structure, drug identifier, dosage record, and the like. In some embodiments, contraindication communication is a contraindication alert communication, indicating the presence of an undesirable or biological effect or contraindication. In some embodiments, contraindication communication is a contraindication-free alert communication, indicating the lack of an undesirable or biological effect or contraindication. In some embodiments, contraindication communication indicates the presence or lack of a desirable or biological effect.

The term “random” refers to a selection of a next node from a starting node, during a traversal, where a probability of selecting the next node out of all connected nodes connected to the starting node is equal to the probability of selecting any of the other nodes connected to the starting node.

The term “biased random” refers to a selection of a next node from a starting node, during a traversal, where a probability of selecting a node directly linked to the starting node as the next node is proportional to a respective edge weight of the node relative to edge weights of other nodes directly linked to the starting node.

The term “side-effect-associated nodes” refers to nodes of a knowledge graph data structure that are associated with physical side effects associated with a given biological data structure. That is, a node, associated with a given biological data structure, may have nodes associated therewith that represent that an identifier (e.g., drug identifier, protein identifier, biomolecule identifier, biological process identifier) associated with the biological data structure may lead to physical side effects for a user identifier associated with the knowledge graph data structure. In some embodiments, side effects are referred to as treatment side effects.

The terms “treatment effectiveness,” “nodes associated with treatment effectiveness,” and “nodes associated with disease amelioration” refer to nodes of a knowledge graph data structure that are associated with effective treatment or disease amelioration associated with a given biological data structure. That is, a node, associated with a given biological data structure, may have nodes associated therewith that represent that an identifier (e.g., drug identifier, protein identifier, biomolecule identifier, biological process identifier) associated with the biological data structure may lead to effective treatment or disease amelioration for a user identifier associated with the knowledge graph data structure.

The term “diagnostic test recommendation” refers to one or more items of data included in a contraindication communication indicative of a recommendation to perform a particular diagnostic test. For example, suppose that a patient has been taking Drug A and a provider is contemplating prescribing Drug B. The biased random walks (e.g., traversals) for Drugs A and B each produce frequent visits to a node representing Protein C which is encoded by Gene C. It is known that the efficacy and side effects of Drug A are influenced by the genotype of Gene C (which affects the configuration of Protein C). The genotype of Gene C and configuration of Protein C are unknown for the patient in this example and knowledge of the genotype and/or protein configuration would determine whether Drug B would be contraindicated for the patient. As a result, embodiments herein may transmit a diagnostic test recommendation to perform a test that would determine the genotype of Gene C and/or configuration of Protein C.

The term “protein identifier” refers to one or more items of data by which a particular protein is uniquely identified. In some embodiments, a protein identifier comprises one or more of ASCII text, encryption keys, identification certificates, a pointer, an IP address, a URL, a MAC address, a memory address, an object signature, a HASH value, or other unique identifier, or combinations thereof.

The term “biomolecule identifier” refers to one or more items of data by which a particular protein is uniquely identified. In some embodiments, a biomolecule identifier comprises one or more of ASCII text, encryption keys, identification certificates, a pointer, an IP address, a URL, a MAC address, a memory address, an object signature, a HASH value, or other unique identifier, or combinations thereof.

The term “biological process identifier” refers to one or more items of data by which a particular biological process is uniquely identified. In some embodiments, a biological process identifier comprises one or more of ASCII text, encryption keys, identification certificates, a pointer, an IP address, a URL, a MAC address, a memory address, an object signature, a HASH value, or other unique identifier, or combinations thereof.

### III. Computer Program Products, Methods, and Computing Entities

Embodiments of the present disclosure may be implemented in various ways, including as computer program products that comprise articles of manufacture. Such computer program products may include one or more software components including, for example, software objects, methods, data structures, or the like. A software component may be coded in any of a variety of programming languages. An illustrative programming language may be a lower-level programming language such as an assembly language associated with a particular hardware architecture and/or operating system platform. A software component comprising assembly language instructions may require conversion into executable machine code by an assembler prior to execution by the hardware architecture and/or platform. Another example programming language may be a higher-level programming language that may be portable across multiple architectures. A software component comprising higher-level programming language instructions may require conversion to an intermediate representation by an interpreter or a compiler prior to execution.

Other examples of programming languages include, but are not limited to, a macro language, a shell or command language, a job control language, a script language, a database query or search language, and/or a report writing language. In one or more embodiments, a software component comprising instructions in one of the foregoing examples of programming languages may be executed directly by an operating system or other software component without having to be first transformed into another form. A software component may be stored as a file or other data storage construct. Software components of a similar type or functionally that are related may be stored together such as, for example, in a particular directory, folder, or library. Software components may be static (e.g., pre-established, or fixed) or dynamic (e.g., created or modified at the time of execution).

A computer program product may include a non-transitory computer-readable storage medium storing applications, programs, program modules, scripts, source code, program code, object code, byte code, compiled code, interpreted code, machine code, executable instructions, and/or the like (also referred to herein as executable instructions, instructions for execution, computer program products, program code, and/or similar terms used herein interchangeably). Such non-transitory computer-readable storage media include all computer-readable media (including volatile and non-volatile media).

In various embodiments, a non-volatile computer-readable storage medium may include a floppy disk, flexible disk, hard disk, solid-state storage (SSS) (e.g., a solid-state drive (SSD)), solid state card (SSC), solid state module (SSM), enterprise flash drive, magnetic tape, or any other non-transitory magnetic medium, and/or the like. A non-volatile computer-readable storage medium may also include a punch card, paper tape, optical mark sheet (or any other physical medium with patterns of holes or other optically recognizable indicia), compact disc read only memory (CD-ROM), compact disc-rewritable (CD-RW), digital versatile disc (DVD), Blu-ray disc (BD), any other non-transitory optical medium, and/or the like. Such a non-volatile computer-readable storage medium may also include read-only memory (ROM), programmable read-only memory (PROM), erasable programmable read-only memory (EPROM), electrically erasable programmable read-only memory (EEPROM), flash memory (e.g., Serial, NAND, NOR, and/or the like), multimedia memory cards (MMC), secure digital (SD) memory cards, SmartMedia cards, CompactFlash (CF) cards, Memory Sticks, and/or the like. Further, a non-volatile computer-readable storage medium may also include conductive-bridging random access memory (CBRAM), phase-change random access memory (PRAM), ferroelectric random-access memory (FeRAM), non-volatile random-access memory (NVRAM), magnetoresistive random-access memory (MRAM), resistive random-access memory (RRAM), Silicon-Oxide-Nitride-Oxide-Silicon memory (SONOS), floating junction gate random access memory (FJG RAM), Millipede memory, racetrack memory, and/or the like.

In various embodiments, a volatile computer-readable storage medium may include random access memory (RAM), dynamic random access memory (DRAM), static random access memory (SRAM), fast page mode dynamic random access memory (FPM DRAM), extended data-out dynamic random access memory (EDO DRAM), synchronous dynamic random access memory (SDRAM), double data rate synchronous dynamic random access memory (DDR SDRAM), double data rate type two synchronous dynamic random access memory (DDR2 SDRAM), double data rate type three synchronous dynamic random access memory (DDR3 SDRAM), Rambus dynamic random access memory (RDRAM), Twin Transistor RAM (TTRAM), Thyristor RAM (T-RAM), Zero-capacitor (Z-RAM), Rambus in-line memory module (RIMM), dual in-line memory module (DIMM), single in-line memory module (SIMM), video random access memory (VRAM), cache memory (including various levels), flash memory, register memory, and/or the like. It will be appreciated that where embodiments are described to use a computer-readable storage medium, other types of computer-readable storage media may be substituted for or used in addition to the computer-readable storage media described above.

As should be appreciated, various embodiments of the present disclosure may also be implemented as methods, apparatus, systems, computing devices, computing entities, and/or the like. As such, embodiments of the present disclosure may take the form of an apparatus, system, computing device, computing entity, and/or the like executing instructions stored on a non-transitory computer-readable storage medium to perform certain steps or operations. Thus, embodiments of the present disclosure may also take the form of an entirely hardware embodiment, an entirely computer program product embodiment, and/or an embodiment that comprises combination of computer program products and hardware performing certain steps or operations.

Embodiments of the present disclosure are described below with reference to block diagrams and flowchart illustrations. Thus, it should be understood that each block of the block diagrams and flowchart illustrations may be implemented in the form of a computer program product, an entirely hardware embodiment, a combination of hardware and computer program products, and/or apparatus, systems, computing devices, computing entities, and/or the like carrying out instructions, operations, steps, and similar words used interchangeably (e.g., the executable instructions, instructions for execution, program code, and/or the like) on a non-transitory computer-readable storage medium for execution. Thus, such embodiments may produce specifically configured machines performing the steps or operations specified in the block diagrams and flowchart illustrations. Accordingly, the block diagrams and flowchart illustrations support various combinations of embodiments for performing the specified instructions, operations, or steps. In some embodiments, retrieval, loading, and execution of code may be performed sequentially such that one instruction is retrieved, loaded, and executed at a time. In some embodiments, retrieval, loading, and/or execution may be performed in parallel such that multiple instructions are retrieved, loaded, and/or executed together.

### IV. Example System Framework

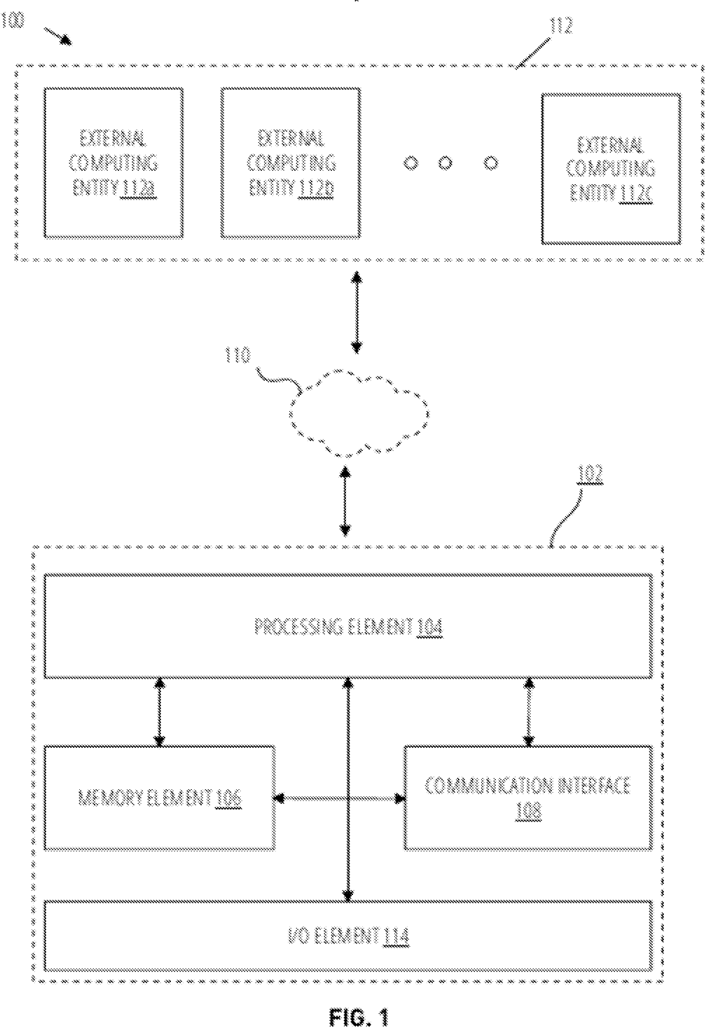

FIG. 1 illustrates an example computing system 100 in accordance with one or more embodiments of the present disclosure. The computing system 100 may include a predictive computing entity 102 and/or one or more external computing entities 112a-c communicatively coupled to the predictive computing entity 102 using one or more wired and/or wireless communication techniques. The predictive computing entity 102 may be specially configured to perform one or more steps/operations of one or more prediction techniques described herein, such as knowledge graph generation and traversal techniques. In some embodiments, the predictive computing entity 102 may include and/or be in association with one or more mobile device(s), desktop computer(s), laptop(s), server(s), cloud computing platform(s), and/or the like. In some embodiments, the predictive computing entity 102 may be configured to receive and/or transmit one or more datasets, objects, and/or the like from and/or to the external computing entities 112a-c to perform one or more steps/operations of one or more prediction techniques described herein.

The external computing entities 112a-c, for example, may include and/or be associated with one or more data centers and/or data processing entities. The data centers, for example, may be associated with one or more data repositories storing data that can, in some circumstances, be processed by the predictive computing entity 102. By way of example, the external computing entities 112a-c may be associated with a plurality of predictive entities associated with a machine learning process. The external computing entities 112a-c, for example, may generate, record, store, and/or maintain historical and/or simulated data associated with the machine learning process. In some embodiments, historical and/or simulated data may include at least a portion of training data leveraged to train a machine learning model. In addition, or alternatively, the external computing entities 112a-c may include one or more data processing entities that may receive, store, and/or have access to one or more machine learning models configured to perform one or more machine learning processes. In such a case, the external computing entities 112a-c may provide access to the machine learning models and/or augment the machine learning models with one or more bias mitigation functionalities described herein.

The predictive computing entity 102 may include, or be in communication with, one or more processing elements 104 (also referred to as processors, processing circuitry, digital circuitry, and/or similar terms used herein interchangeably) that communicate with other elements within the predictive computing entity 102 via a bus, for example. As will be understood, the predictive computing entity 102 may be embodied in a number of different ways. The predictive computing entity 102 may be configured for a particular use or configured to execute instructions stored in volatile or non-volatile media or otherwise accessible to the processing element 104. As such, whether configured by hardware or computer program products, or by a combination thereof, the processing element 104 may be capable of performing steps or operations according to embodiments of the present disclosure when configured accordingly.

In one embodiment, the predictive computing entity 102 may further include, or be in communication with, one or more memory elements 106. The memory element 106 may be used to store at least portions of the databases, database instances, database management systems, data, applications, programs, program modules, scripts, source code, object code, byte code, compiled code, interpreted code, machine code, executable instructions, and/or the like being executed by, for example, the processing element 104. Thus, the databases, database instances, database management systems, data, applications, programs, program modules, scripts, source code, object code, byte code, compiled code, interpreted code, machine code, executable instructions, and/or the like may be used to control certain aspects of the operation of the predictive computing entity 102 with the assistance of the processing element 104.

As indicated, in one embodiment, the predictive computing entity 102 may also include one or more communication interfaces 108 for communicating with various computing entities such as the external computing entities 112a-c, such as by communicating data, content, information, and/or similar terms used herein interchangeably that may be transmitted, received, operated on, processed, displayed, stored, and/or the like.

The computing system 100 may include one or more input/output (I/O) element(s) 114 for communicating with one or more users. An I/O element 114, for example, may include one or more user interfaces for providing and/or receiving information from one or more users of the computing system 100. The I/O element 114 may include one or more tactile interfaces (e.g., keypads, touch screens, etc.), one or more audio interfaces (e.g., microphones, speakers, etc.), visual interfaces (e.g., display devices, etc.), and/or the like. The I/O element 114 may be configured to receive user input through one or more of the user interfaces from a user of the computing system 100 and provide data to a user through the user interfaces.

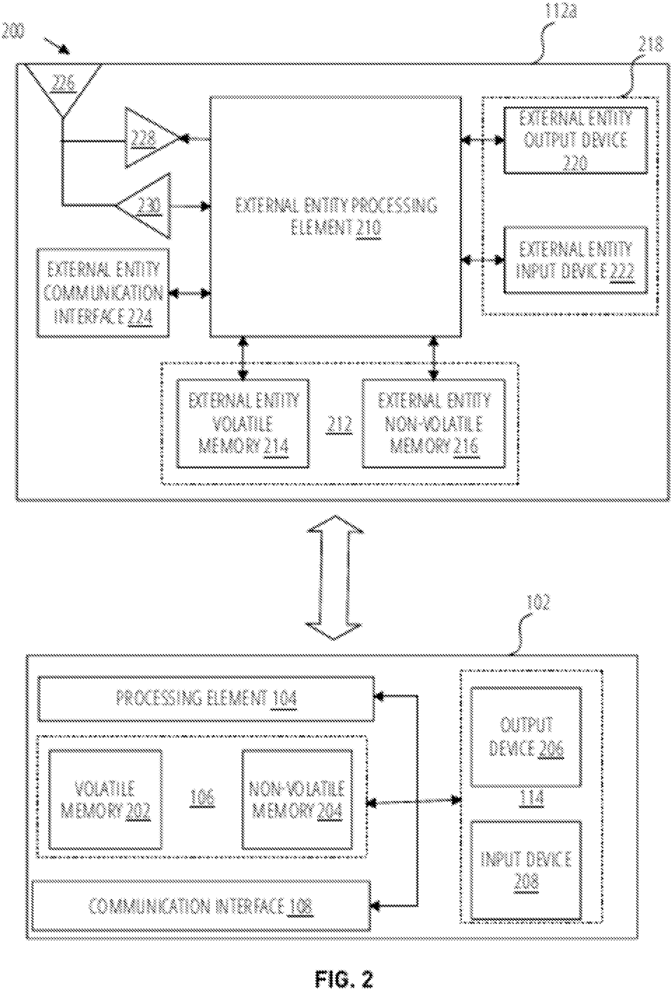

FIG. 2 is a schematic diagram showing a system computing architecture 200 in accordance with some embodiments discussed herein. In some embodiments, the system computing architecture 200 may include the predictive computing entity 102 and/or the external computing entity 112a of the computing system 100. The predictive computing entity 102 and/or the external computing entity 112a may include a computing apparatus, a computing device, and/or any form of computing entity configured to execute instructions stored on a computer-readable storage medium to perform certain steps or operations.

The predictive computing entity 102 may include a processing element 104, a memory element 106, a communication interface 108, and/or one or more I/O elements 114 that communicate within the predictive computing entity 102 via internal communication circuitry such as a communication bus, and/or the like.

The processing element 104 may be embodied as one or more complex programmable logic devices (CPLDs), microprocessors, multi-core processors, coprocessing entities, application-specific instruction-set processors (ASIPs), microcontrollers, and/or controllers. Further, the processing element 104 may be embodied as one or more other processing devices or circuitry including, for example, a processor, one or more processors, various processing devices and/or the like. The term circuitry may refer to an entirely hardware embodiment or a combination of hardware and computer program products. Thus, the processing element 104 may be embodied as integrated circuits, application specific integrated circuits (ASICs), field programmable gate arrays (FPGAs), programmable logic arrays (PLAs), hardware accelerators, digital circuitry, and/or the like.

The memory element 106 may include volatile memory 202 and/or non-volatile memory 204. The memory element 106, for example, may include volatile memory 202 (also referred to as volatile storage media, memory storage, memory circuitry and/or similar terms used herein interchangeably). In one embodiment, a volatile memory 202 may include random access memory (RAM), dynamic random access memory (DRAM), static random access memory (SRAM), fast page mode dynamic random access memory (FPM DRAM), extended data-out dynamic random access memory (EDO DRAM), synchronous dynamic random access memory (SDRAM), double data rate synchronous dynamic random access memory (DDR SDRAM), double data rate type two synchronous dynamic random access memory (DDR2 SDRAM), double data rate type three synchronous dynamic random access memory (DDR3 SDRAM), Rambus dynamic random access memory (RDRAM), Twin Transistor RAM (TTRAM), Thyristor RAM (T-RAM), Zero-capacitor (Z-RAM), Rambus in-line memory module (RIMM), dual in-line memory module (DIMM), single in-line memory module (SIMM), video random access memory (VRAM), cache memory (including various levels), flash memory, register memory, and/or the like. It will be appreciated that where embodiments are described to use a computer-readable storage medium, other types of computer-readable storage media may be substituted for or used in addition to the computer-readable storage media described above.

The memory element 106 may include non-volatile memory 204 (also referred to as non-volatile storage, memory, memory storage, memory circuitry and/or similar terms used herein interchangeably). In one embodiment, the non-volatile memory 204 may include one or more non-volatile storage or memory media, including, but not limited to, hard disks, ROM, PROM, EPROM, EEPROM, flash memory, MMCs, SD memory cards, Memory Sticks, CBRAM, PRAM, FeRAM, NVRAM, MRAM, RRAM, SONOS, FJG RAM, Millipede memory, racetrack memory, and/or the like.

In one embodiment, a non-volatile memory 204 may include a floppy disk, flexible disk, hard disk, solid-state storage (SSS) (e.g., a solid-state drive (SSD)), solid state card (SSC), solid state module (SSM), enterprise flash drive, magnetic tape, or any other non-transitory magnetic medium, and/or the like. A non-volatile memory 204 may also include a punch card, paper tape, optical mark sheet (or any other physical medium with patterns of holes or other optically recognizable indicia), compact disc read only memory (CD-ROM), compact disc-rewritable (CD-RW), digital versatile disc (DVD), Blu-ray disc (BD), any other non-transitory optical medium, and/or the like. Such a non-volatile memory 204 may also include read-only memory (ROM), programmable read-only memory (PROM), erasable programmable read-only memory (EPROM), electrically erasable programmable read-only memory (EEPROM), flash memory (e.g., Serial, NAND, NOR, and/or the like), multimedia memory cards (MMC), secure digital (SD) memory cards, SmartMedia cards, CompactFlash (CF) cards, Memory Sticks, and/or the like. Further, a non-volatile computer-readable storage medium may also include conductive-bridging random access memory (CBRAM), phase-change random access memory (PRAM), ferroelectric random-access memory (FeRAM), non-volatile random-access memory (NVRAM), magnetoresistive random-access memory (MRAM), resistive random-access memory (RRAM), Silicon-Oxide-Nitride-Oxide-Silicon memory (SONOS), floating junction gate random access memory (FJG RAM), Millipede memory, racetrack memory, and/or the like.

As will be recognized, the non-volatile memory 204 may store databases, database instances, database management systems, data, applications, programs, program modules, scripts, source code, object code, byte code, compiled code, interpreted code, machine code, executable instructions, and/or the like. The term database, database instance, database management system, and/or similar terms used herein interchangeably may refer to a collection of records or data that is stored in a computer-readable storage medium using one or more database models, such as a hierarchical database model, network model, relational model, entity-relationship model, object model, document model, semantic model, graph model, and/or the like.

The memory element 106 may include a non-transitory computer-readable storage medium for implementing one or more aspects of the present disclosure including as a computer-implemented method configured to perform one or more steps/operations described herein. For example, the non-transitory computer-readable storage medium may include instructions that when executed by a computer (e.g., processing element 104), cause the computer to perform one or more steps/operations of the present disclosure. For instance, the memory element 106 may store instructions that, when executed by the processing element 104, configure the predictive computing entity 102 to perform one or more step/operations described herein.

Embodiments of the present disclosure may be implemented in various ways, including as computer program products that comprise articles of manufacture. Such computer program products may include one or more software components including, for example, software objects, methods, data structures, or the like. A software component may be coded in any of a variety of programming languages. An illustrative programming language may be a lower-level programming language such as an assembly language associated with a particular hardware framework and/or operating system platform. A software component comprising assembly language instructions may require conversion into executable machine code by an assembler prior to execution by the hardware framework and/or platform. Another example programming language may be a higher-level programming language that may be portable across multiple frameworks. A software component comprising higher-level programming language instructions may require conversion to an intermediate representation by an interpreter or a compiler prior to execution.

Other examples of programming languages include, but are not limited to, a macro language, a shell or command language, a job control language, a script language, a database query, or search language, and/or a report writing language. In one or more example embodiments, a software component comprising instructions in one of the foregoing examples of programming languages may be executed directly by an operating system or other software component without having to be first transformed into another form. A software component may be stored as a file or other data storage construct. Software components of a similar type or functionally related may be stored together such as in a particular directory, folder, or library. Software components may be static (e.g., pre-established, or fixed) or dynamic (e.g., created or modified at the time of execution).

The predictive computing entity 102 may be embodied by a computer program product include non-transitory computer-readable storage medium storing applications, programs, program modules, scripts, source code, program code, object code, byte code, compiled code, interpreted code, machine code, executable instructions, and/or the like (also referred to herein as executable instructions, instructions for execution, computer program products, program code, and/or similar terms used herein interchangeably). Such non-transitory computer-readable storage media include all computer-readable media such as the volatile memory 202 and/or the non-volatile memory 204.

The predictive computing entity 102 may include one or more I/O elements 114. The I/O elements 114 may include one or more output devices 206 and/or one or more input devices 208 for providing and/or receiving information with a user, respectively. The output devices 206 may include one or more sensory output devices such as one or more tactile output devices (e.g., vibration devices such as direct current motors, and/or the like), one or more visual output devices (e.g., liquid crystal displays, and/or the like), one or more audio output devices (e.g., speakers, and/or the like), and/or the like. The input devices 208 may include one or more sensory input devices such as one or more tactile input devices (e.g., touch sensitive displays, push buttons, and/or the like), one or more audio input devices (e.g., microphones, and/or the like), and/or the like.

In addition, or alternatively, the predictive computing entity 102 may communicate, via a communication interface 108, with one or more external computing entities such as the external computing entity 112a. The communication interface 108 may be compatible with one or more wired and/or wireless communication protocols.

For example, such communication may be executed using a wired data transmission protocol, such as fiber distributed data interface (FDDI), digital subscriber line (DSL), Ethernet, asynchronous transfer mode (ATM), frame relay, data over cable service interface specification (DOCSIS), or any other wired transmission protocol. In addition, or alternatively, the predictive computing entity 102 may be configured to communicate via wireless external communication using any of a variety of protocols, such as general packet radio service (GPRS), Universal Mobile Telecommunications System (UMTS), Code Division Multiple Access 2000 (CDMA2000), CDMA2000 1X (1xRTT), Wideband Code Division Multiple Access (WCDMA), Global System for Mobile Communications (GSM), Enhanced Data rates for GSM Evolution (EDGE), Time Division-Synchronous Code Division Multiple Access (TD-SCDMA), Long Term Evolution (LTE), Evolved Universal Terrestrial Radio Access Network (E-UTRAN), Evolution-Data Optimized (EVDO), High Speed Packet Access (HSPA), High-Speed Downlink Packet Access (HSDPA), IEEE 802.9 (Wi-Fi), Wi-Fi Direct, 802.16 (WiMAX), ultra-wideband (UWB), infrared (IR) protocols, near field communication (NFC) protocols, Wibree, Bluetooth protocols, wireless universal serial bus (USB) protocols, and/or any other wireless protocol.

The external computing entity 112a may include an external entity processing element 210, an external entity memory element 212, an external entity communication interface 224, and/or one or more external entity I/O elements 218 that communicate within the external computing entity 112a via internal communication circuitry such as a communication bus, and/or the like.

The external entity processing element 210 may include one or more processing devices, processors, and/or any other device, circuitry, and/or the like described with reference to the processing element 104. The external entity memory element 212 may include one or more memory devices, media, and/or the like described with reference to the memory element 106. The external entity memory element 212, for example, may include at least one external entity volatile memory 214 and/or external entity non-volatile memory 216. The external entity communication interface 224 may include one or more wired and/or wireless communication interfaces as described with reference to communication interface 108.

In some embodiments, the external entity communication interface 224 may be supported by one or more radio circuitries. For instance, the external computing entity 112a may include an antenna 226, a transmitter 228 (e.g., radio), and/or a receiver 230 (e.g., radio).

Signals provided to and received from the transmitter 228 and the receiver 230, correspondingly, may include signaling information/data in accordance with air interface standards of applicable wireless systems. In this regard, the external computing entity 112a may be capable of operating with one or more air interface standards, communication protocols, modulation types, and access types. More particularly, the external computing entity 112a may operate in accordance with any of a number of wireless communication standards and protocols, such as those described above regarding the predictive computing entity 102.

Via these communication standards and protocols, the external computing entity 112a may communicate with various other entities using means such as Unstructured Supplementary Service Data (USSD), Short Message Service (SMS), Multimedia Messaging Service (MMS), Dual-Tone Multi-Frequency Signaling (DTMF), and/or Subscriber Identity Module Dialer (SIM dialer). The external computing entity 112a may also download changes, add-ons, and updates, for instance, to its firmware, software (e.g., including executable instructions, applications, program modules), operating system, and/or the like.

According to one embodiment, the external computing entity 112a may include location determining embodiments, devices, modules, functionalities, and/or the like. For example, the external computing entity 112a may include outdoor positioning embodiments, such as a location module adapted to acquire, for example, latitude, longitude, altitude, geocode, course, direction, heading, speed, universal time (UTC), date, and/or various other information/data. In one embodiment, the location module may acquire data such as ephemeris data, by identifying the number of satellites in view and the relative positions of those satellites (e.g., using global positioning systems (GPS)). The satellites may be a variety of different satellites, including Low Earth Orbit (LEO) satellite systems, Department of Defense (DOD) satellite systems, the European Union Galileo positioning systems, the Chinese Compass navigation systems, Indian Regional Navigational satellite systems, and/or the like. This data may be collected using a variety of coordinate systems, such as the Decimal Degrees (DD); Degrees, Minutes, Seconds (DMS); Universal Transverse Mercator (UTM); Universal Polar Stereographic (UPS) coordinate systems; and/or the like. Alternatively, the location information/data may be determined by triangulating a position of the external computing entity 112a in connection with a variety of other systems, including cellular towers, Wi-Fi access points, and/or the like. Similarly, the external computing entity 112a may include indoor positioning embodiments, such as a location module adapted to acquire, for example, latitude, longitude, altitude, geocode, course, direction, heading, speed, time, date, and/or various other information/data. Some of the indoor systems may use various position or location technologies including RFID tags, indoor beacons or transmitters, Wi-Fi access points, cellular towers, nearby computing devices (e.g., smartphones, laptops) and/or the like. For instance, such technologies may include the iBeacons, Gimbal proximity beacons, Bluetooth Low Energy (BLE) transmitters, NFC transmitters, and/or the like. These indoor positioning embodiments may be used in a variety of settings to determine the location of someone or something to within inches or centimeters.

The external entity I/O elements 218 may include one or more external entity output devices 220 and/or one or more external entity input devices 222 that may include one or more sensory devices described herein with reference to the I/O elements 114. In some embodiments, the external entity I/O element 218 may include a user interface (e.g., a display, speaker, and/or the like) and/or a user input interface (e.g., keypad, touch screen, microphone, and/or the like) that may be coupled to the external entity processing element 210.

For example, the user interface may be a user application, browser, and/or similar words used herein interchangeably executing on and/or accessible via the external computing entity 112a to interact with and/or cause the display, announcement, and/or the like of information/data to a user. The user input interface may include any of a number of input devices or interfaces allowing the external computing entity 112a to receive data including, as examples, a keypad (hard or soft), a touch display, voice/speech interfaces, motion interfaces, and/or any other input device. In some embodiments including a keypad, the keypad may include (or cause display of) the conventional numeric (0-9) and related keys (#, *, and/or the like), and other keys used for operating the external computing entity 112a and may include a full set of alphabetic keys or set of keys that may be activated to provide a full set of alphanumeric keys. In addition to providing input, the user input interface may be used, for example, to activate or deactivate certain functions, such as screen savers, sleep modes, and/or the like.

### V. Example System Operations

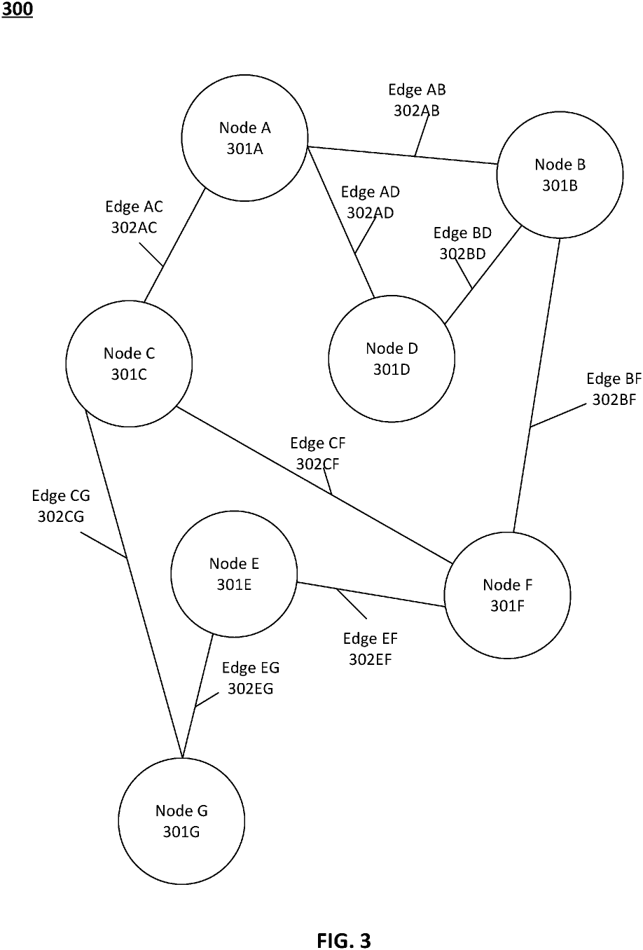

FIG. 3 depicts an example knowledge graph data structure 300 in accordance with some embodiments discussed herein. Shown in FIG. 3, a knowledge graph data structure 300 may include multiple nodes, Node A 301A, Node B 301B, Node C 301C, Node D 301D, Node E 301E, Node F 301F, Node G 301G, and multiple edges Edge AB 302AB, Edge AC 302AC, Edge AD 302AD, Edge BD 302BD, Edge BF 302BF, Edge CF 302CF, Edge CG 302CG, Edge EF 302EF, and Edge EG 302EG.

Also shown in FIG. 3, each edge connects two nodes. For example, Node A 301A and Node B 301B are connected by Edge AB 302AB, which represents a relationship between the two nodes. Edge AC 302AC connects Node A 301A and Node C 301C and represents a relationship therebetween. Edge AD 302AD connects Node A 301A and Node D 301D and represents a relationship therebetween. Edge BD 302BD connects Node B 301B and Node D 301D and represents a relationship therebetween. Edge BF 302BF connects Node B 301B and Node F 301F and represents a relationship therebetween. Edge CF 302CF connects Node C 301C and Node F 301F and represents a relationship therebetween. Edge CG 302CG connects Node C 301C and Node G 301G and represents a relationship therebetween. Edge EF 302EF connects Node E 301E and Node F01F. Edge EG 302EG connects Node E 301E and Node G 301G and represents a relationship therebetween.

Each of edges Edge AB 302AB, Edge AC 302AC, Edge AD 302AD, Edge BD 302BD, Edge BF 302BF, Edge CF 302CF, Edge CG 302CG, Edge EF 302EF, and Edge EG 302EG may be associated with a respective edge weight (not shown in FIG. 3) representative of a significance of the relationship associated therewith.

In some embodiments, the knowledge graph data structure 300 is a biochemical knowledge graph (BKG). In some embodiments, the knowledge graph data structure 300 is a graph-based representation of the physiological state of an individual; the nodes represent proteins and other biomolecules as well as other biological processes and the edges represent relationships among the connected nodes.

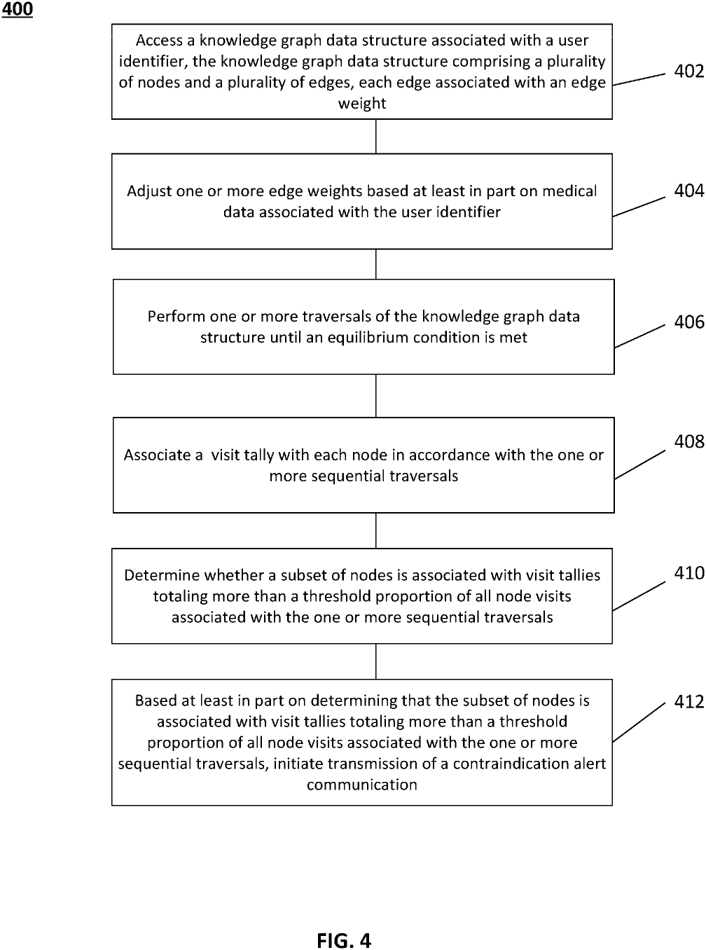

FIG. 4 is a flowchart showing an example of a process 400 for generating contraindication communications in accordance with some embodiments discussed herein. The process 400 leverages new knowledge graph generation and traversal techniques to generate contraindication communications that are specifically tailored to a user identifier. The bias mitigation techniques may be implemented by one or more computing devices, entities, and/or systems described herein. For example, via the various steps/operations of the process 400 (and process 500 described below), the computing system 100, 200 may leverage the knowledge graph generation and traversal techniques to overcome the various limitations with conventional contraindication identification processes that (i) lack specificity with respect to individual patients and/or (ii) fail to account for individual differences unique to each individual patient.

FIG. 4 illustrates an example process 400 for explanatory purposes. Although the example process 400 depicts a particular sequence of steps/operations, the sequence may be altered without departing from the scope of the present disclosure. For example, some of the steps/operations depicted may be performed in parallel or in a different sequence that does not materially impact the function of the process 400. In other examples, different components of an example device or system that implements the process 400 may perform functions at substantially the same time or in a specific sequence.

The process 400 includes, at step/operation 402, accessing a knowledge graph data structure. For example, the computing system 100, 200 may access the knowledge graph data structure 300. In some embodiments, the knowledge graph data structure is a graph-based representation associated with a user identifier. The knowledge graph data structure includes multiple nodes each representing a biological data structure, and multiple edges. Each edge connects two nodes, each edge represents a relationship associated with the two nodes connected by the edge, each edge is associated with an edge weight, and each edge weight is initially a default edge weight.

The process 400 includes, at step/operation 404, adjusting one or more edge weights based on medical data associated with the user identifier. For example, the computing system 100, 200 may adjust one or more edge weights based on medical data associated with the user identifier.

In some embodiments, the default edge weights of the knowledge graph data structure are defined according to values obtained from research studies involving multiple subjects (who may or may not be representative of larger population averages). In process 400, the edge weights may be adjusted based on available information (e.g., medical data) known about the patient who would potentially receive a new drug. Medical data may include, without limitation, clinical notes (e.g., comorbidities, current medications), lab values, insurance claims, demographic information, medical images, and/or sensor data (e.g., continuous glucose data, or acceleration data indicative of physical activity). Some embodiments adjust edge weights based on temporal aspects of the data (e.g., the date or timestamp associated with the data).

In some embodiments, alteration or adjustment of edge weights involves mapping words and data values to edge weights or adjustments to default edge weights. For example, suppose that medical notes indicate that the patient has type-2 diabetes characterized by decreased insulin sensitivity. Insulin sensitivity refers to the degree to which a given concentration of insulin decreases blood glucose level. Accordingly, the predictive computing entity 102 may decrease the edge weight between a node representative of blood glucose and a node representative of insulin where the edge was directional (from insulin to glucose), the relationship type was “decreases,” and the edge weight was proportional to the amount of the decrease. The amount of decrease in the edge weight between the glucose and insulin nodes could be further refined if the patient’s medical record included glucose and insulin values indicative of the patient’s insulin sensitivity. Additionally, edge weights adjusted according to the effect of a drug may be adjusted according to the dosage or contemplated dosage of the drug. In some embodiments, neural network encoders in combination with the knowledge graph data structure traversal described herein, as well as back-propagation techniques, may be used to set or adjust the edge weights.

It will be appreciated that, embodiments herein may include a knowledge graph data structure that is generated for a particular user identifier using default edge weights or edge weights generated based on population averages. That is, in certain embodiments, adjusting the edge weights based on the medical data associated with the particular user identifier is optional.

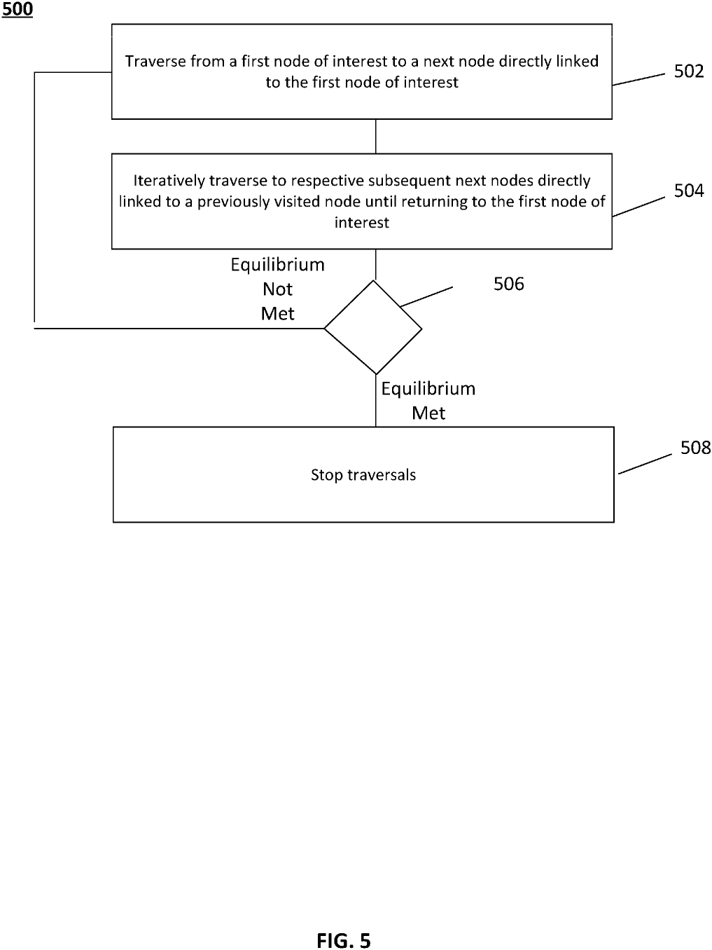

The process 400 includes, at step/operation 406, performing one or more sequential traversals of the knowledge graph data structure until an equilibrium condition is met. For example, the computing system 100, 200 may perform one or more sequential traversals of the knowledge graph data structure 300 until an equilibrium condition is met. In some embodiments, traversals may include biased random walks that start at a node representing a potential drug that might be given to the patient. In some embodiments, wherein the equilibrium condition is met when a variation in a proportion of total visits received by each node caused by an additional traversal falls below a variation threshold. Traversals are further described with respect to FIG. 5 below.

The process 400 includes, at step/operation 408, associating a first visit tally with each node in accordance with the one or more sequential traversals. For example, the computing system 100, 200 may associate a first visit tally with each node in accordance with the one or more sequential traversals of knowledge graph data structure 300. It will be appreciated that, while the description herein references knowledge graph data structure 300 with respect to the traversal and other processes, references to knowledge graph data structure 300 are not intended to be limiting and are for example purposes only. Other knowledge graph data structure sizes and configurations are within the scope of the present disclosure.

The process 400 includes, at step/operation 410, determining whether a subset of nodes is associated with visit tallies totaling more than a threshold proportion of all node visits associated with the one or more sequential traversals. For example, the computing system 100 may determine whether a subset of nodes is associated with visit tallies totaling more than a threshold proportion of all node visits associated with the one or more sequential traversals, where the subset of nodes represent a biological effect associated with a biological data structure associated with the node of interest.

For example, the subset of nodes may collectively be associated with an biological physiological state such as a side effect or a desirable state such as improved health. If the subset is associated with an biological side effect, giving the patient the contemplated drug will likely cause an undesirable side effect and thus be contraindicated. In some embodiments, relative costs and benefits associated with the different subsets of visited nodes may factor into a contraindication communication. In some embodiments, the overall chemical profile of the patient after receiving the contemplated drug (as predicted by traversing the knowledge graph data structure) may be referred to as the treatment profile.

The process 400 includes, at step/operation 412, based on determining that the subset of nodes is associated with first visit tallies totaling more than a threshold proportion of all node visits associated with the one or more sequential traversals, initiating transmission of a contraindication alert communication comprising representation of the biological effect for the user identifier. For example, the computing system 100 may initiate transmission of a contraindication alert communication comprising representation of the biological effect for the user identifier, based on determining that first subset of nodes is associated with visit tallies totaling more than a threshold proportion of all node visits associated with the one or more sequential traversals. In some embodiments, the contraindication alert communication includes a diagnostic test recommendation.

FIG. 5 is a flowchart showing an example of a process 500 for traversing a knowledge graph data structure in accordance with some embodiments discussed herein. FIG. 5 illustrates an example process 500 for explanatory purposes. Although the example process 500 depicts a particular sequence of steps/operations, the sequence may be altered without departing from the scope of the present disclosure. For example, some of the steps/operations depicted may be performed in parallel or in a different sequence that does not materially impact the function of the process 500. In other examples, different components of an example device or system that implements the process 500 may perform functions at substantially the same time or in a specific sequence.

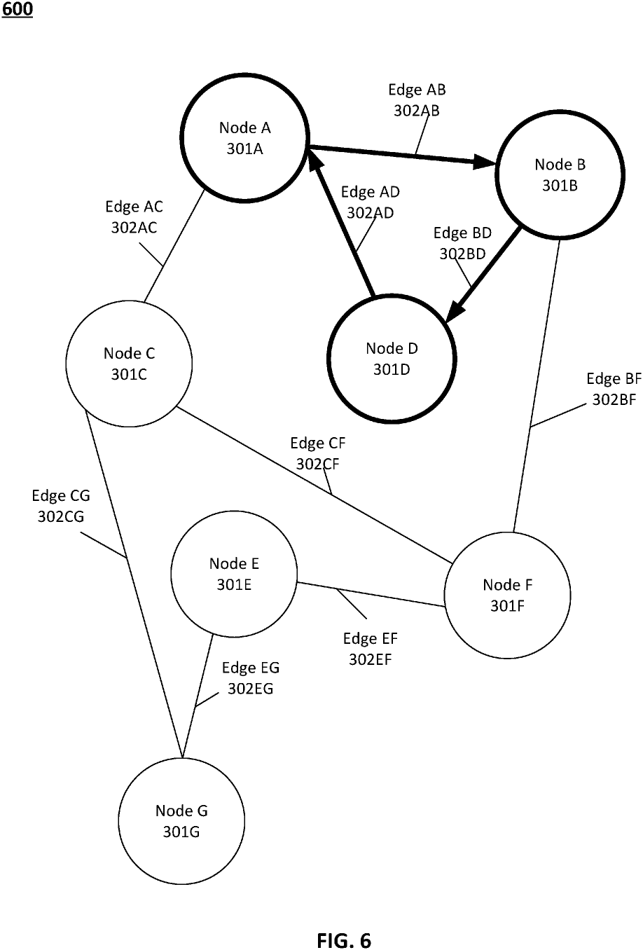

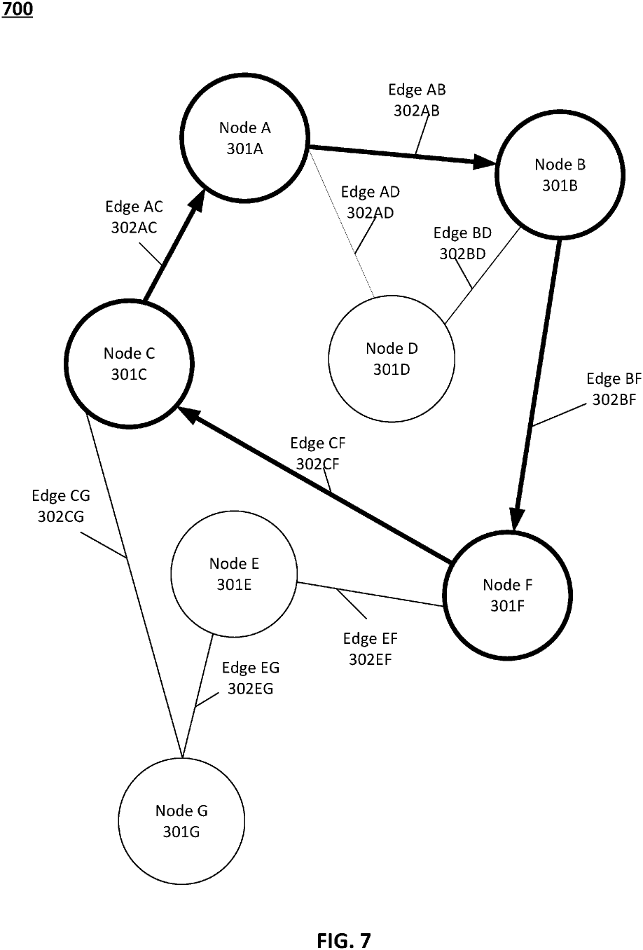

The process 500 includes, at step/operation 502, traversing from a first node of interest to a second node directly linked to the first node of interest. For example, the computing system 100, 200 may start at a first node of interest and traverse to a second node directly linked to the first node of interest (e.g., traversals can be further understood with reference to FIGS. 6 and 7 described below).

The process 500 includes, at step/operation 504, iteratively traversing to respective subsequent nodes directly linked to a previously visited node (e.g., the second node is a subsequent node directly linked to the previously visited node, which is the first node of interest) until returning to the first node of interest. For example, the computing system 100 may iteratively traverse to respective subsequent nodes directly linked to a previously visited node until returning to the first node of interest. In some embodiments, a subsequent node becomes a previously visited node upon traversal away from the subsequent node.

The process 500 includes, at step/operation 506, determining whether an equilibrium condition is met. For example, the computing system 100, 200 may determine whether the equilibrium condition is met. If the equilibrium condition is met, the process 500 proceeds to step/operation 508 (e.g., stops traversing). If the equilibrium condition is not met, the process 500 proceeds to step/operation 502.

In some embodiments, a random walk may refer to a series of connected nodes in a knowledge graph data structure. A random walk starts at a node of interest in terms of its influence on the system (e.g., user identifier) represented by the knowledge graph data structure. In some embodiments herein, random walks start at a first node representing a drug that may be administered to a patient whose biochemical state is represented by the knowledge graph data structure with edge weights adjusted as described herein. Once on the first node, a random walk selects a subsequent node randomly from the set of nodes directly linked to the first node. If the random walk is unbiased, the likelihood of selecting each of the connected nodes is equal to the likelihood or probability of selecting any of the other connected nodes. In a biased random walk, the likelihoods are not equal. In a biased random walk, and in some embodiments herein, the probability of selecting any connected node is proportional to the edge weight of the edge connecting the nodes relative to the edge weights of the other connected nodes. Thus, adjusting the edge weights herein may influence the likelihood of the nodes that will be visited on any given biased random walk. The biased random walk proceeds to the selected node and repeats the biased random selection of subsequent nodes that are connected to it. Alternatively to selecting a subsequent node, a random walk may randomly return to the first node from any subsequent node. An individual random walk ends when the walk returns to the first node. Biased random walks are continuously performed until the visit state has reached equilibrium. Equilibrium is achieved when the variation in the proportion of total visits received by each node caused by an additional random walk falls below a threshold. The proportion of total visits will reflect the edge weights.

FIG. 6 is an operational example 600 of a knowledge graph data structure traversal in accordance with some embodiments discussed herein. Shown in FIG. 6, a first node or node of interest may be Node A 301A. Starting at Node A 301A, a biased random walk or traversal may continue to a second node, Node B 301B, based on a proportionality of an edge weight (not shown) associated with Edge AB 302AB as compared to an edge weight (not shown) associated with Edge AD 302AD. The biased random walk or traversal may then continue to a subsequent node, Node D 301D, based on a proportionality of an edge weight (not shown) associated with Edge AB 302AB, Edge BF 302BF, and Edge BD 302BD (comparatively). The biased random walk or traversal may then continue to a subsequent node, Node A 301A, based on a proportionality of an edge weight (not shown) associated with Edge AD 302AD as compared to Edge BD 302BD. Because the biased random walk or traversal has returned to Node A 301A (the node of interest, first node), the biased random walk or traversal is complete. Based on the traversal depicted in FIG. 6, each of Node A 301A, Node B 301B, and Node D 301D may be associated with a visit tally of one (1) and the traversal may be associated with a total visit tally of three (3).

FIG. 7 is an operational example 700 of a knowledge graph data structure traversal in accordance with some embodiments discussed herein. Shown in FIG. 7, a first node or node of interest may be Node A 301A. Starting at Node A 301A, a biased random walk or traversal may continue to a second node, Node B 301B, based on a proportionality of an edge weight (not shown) associated with Edge AB 302AB as compared to an edge weight (not shown) associated with Edge AD 302AD. The biased random walk or traversal may then continue to a subsequent node, Node F 301F, based on a proportionality of an edge weight (not shown) associated with Edge AB 302AB, Edge BF 302BF, and Edge BD 302BD (comparatively). The biased random walk or traversal may then continue to a subsequent node, Node C 301C, based on a proportionality of an edge weight (not shown) associated with Edge CF 302CF as compared to Edge BF 302BF.

The biased random walk or traversal may then continue to a subsequent node, Node A 301A, based on a proportionality of an edge weight (not shown) associated with Edge AC 302AC as compared to Edge CF 302CF. Because the biased random walk or traversal has returned to Node A 301A (the node of interest, first node), the biased random walk or traversal is complete. Based on the traversal depicted in FIG. 7, each of Node A 301A, Node B 301B, Node C 301C, and Node F 301F may be associated with a visit tally of one (1) and the traversal may be associated with a total visit tally of four (4).

In some embodiments, biased random walks or traversals may start from two or more nodes of interested (e.g., alternative drugs) and a contraindication communication may include a selection of the alternative drug that has the most desirable treatment profile. Selection criteria may include, for example, minimizing the number of visits to side-effect-associated nodes or maximizing the ratio of treatment effectiveness (as determined by the number of visits to nodes associated with disease amelioration) to treatment side effects (as determined by the number of visits to side-effect-associated nodes).

In some embodiments, if a contraindication or undesirable biological effect is associated with a first drug identifier and a first dosage record, the processes set forth herein may be repeated with a second dosage record to determine whether the contraindication or undesirable biological effect may be eliminated by adjusting a dosage.

Thus, as has been discussed, embodiments herein provide technological improvements over existing methodologies for identifying possible contraindications for particular individual patients by employing individualized graph-based representations of a physiological state of a particular patient and systematically and programmatically traversing the graph-based representation to identify possible contraindications for any possible combination of drug or condition specific to the patient. Further, embodiments of the present disclosure present innovative traversal techniques associated with a knowledge graph data structure specifically generated for a given user identifier so that treatment selection for the user identifier is personalized according to physiological characteristics of the user identifier. Such approaches less intense computing resources and time such that a result is produced after the underlying data or conditions are no longer present or relevant to the analysis.

### VI. Conclusion

Many modifications and other embodiments will come to mind to one skilled in the art to which this present disclosure pertains having the benefit of the teachings presented in the foregoing descriptions and the associated drawings. Therefore, it is to be understood that the present disclosure is not to be limited to the specific embodiments disclosed and that modifications and other embodiments are intended to be included within the scope of the appended claims. Although specific terms are employed herein, they are used in a generic and descriptive sense only and not for purposes of limitation.

### VII. EXAMPLES

Example 1. A computing apparatus comprising memory and one or more processors communicatively coupled to the memory. The one or more processors are configured to access a knowledge graph data structure that comprises (i) a graph-based representation associated with a user identifier, (ii) a plurality of nodes, wherein each node represents a biological data structure, (iii) a plurality of edges, wherein each of the plurality of edges (a) connects two respective nodes, (b) represents a relationship associated with the two nodes connected by the edge, (c) is associated with an edge weight, and (d) is initially associated with a respective default edge weight. The one or more processors are further configured to adjust one or more edge weights based at least in part on medical data associated with the user identifier. The one or more processors are further configured to perform one or more sequential traversals of the knowledge graph data structure, starting at a node of interest, until an equilibrium condition is met when a variation in a proportion of total visits received by each of the plurality of nodes caused by the one or more sequential traversals falls below a variation threshold. The one or more processors are further configured to determine whether a subset of the plurality of nodes is associated with visit tallies totaling more than a threshold associated with the proportion of total visits, wherein the subset of nodes represents a biological effect associated with the node of interest. The one or more processors are further configured to, based at least in part on determining that the subset of the plurality of nodes is associated with the visit tallies, initiate transmission of a contraindication alert communication.

Example 2. The computing apparatus of example 1, wherein performing the one or more sequential traversals of the knowledge graph data structure until the equilibrium condition is met comprises traversing from a first node to a second node directly linked to the first node, and iteratively traversing to subsequent nodes until returning to the first node.

Example 3. The computing apparatus of example 1 or 2, wherein the contraindication alert communication comprises representation of the biological effect for the user identifier.

Example 4. The computing apparatus of any of the preceding examples, wherein the node of interest is a first node of interest associated with the first biological data structure comprising a first drug identifier and a first dosage record.

Example 5. The computing apparatus of any of the preceding examples, wherein a second node of interest is associated with a second biological data structure comprising the first drug identifier and a second dosage record.

Example 6. The computing apparatus of any of the preceding examples, wherein the one or more processors are further configured to perform one or more additional sequential traversals of the knowledge graph data structure until the equilibrium condition is met, by traversing from the second node of interest to a next node directly linked to the second node of interest, and iteratively traversing to respective subsequent next nodes directly linked to a previously visited node until returning to the second node of interest. The one or more processors are further configured to associate a second visit tally with each node in accordance with the one or more sequential traversals. The one or more processors are further configured determine whether a second subset of nodes is associated with second visit tallies totaling more than a second threshold proportion of all node visits associated with the one or more additional sequential traversals, wherein the second subset of nodes represents a second biological effect associated with a second biological data structure associated with the second node of interest. The one or more processors are further configured to, based at least in part on determining that the second subset of nodes is not associated with second visit tallies totaling more than a threshold proportion of all node visits associated with the one or more additional sequential traversals, initiate transmission of a contraindication alert communication comprising representation of a lack of the second biological effect for the user identifier.

Example 7. The computing apparatus of any of the preceding examples, wherein selecting the next node to which to traverse from the first node of interest is performed randomly.

Example 8. The computing apparatus of any of the preceding examples, wherein a first probability of selecting a node directly linked to the node of interest as the next node is proportional to a respective edge weight of the node relative to edge weights of other nodes directly linked to the node of interest.

Example 9. The computing apparatus of any of the preceding examples, wherein selecting the subsequent next node to which to traverse from the previously visited node is performed randomly.

Example 10. The computing apparatus of any of the preceding examples, wherein a second probability of selecting a node directly linked to the previously visited node is proportional to a respective edge weight of the node relative to edge weights of other nodes directly linked to the previously visited node.

Example 11. The computing apparatus of any of the preceding examples, wherein the medical data associated with the user identifier comprises one or more of clinical notes data, comorbidities data, current medications data, lab values data, insurance claims data, demographic data, medical images data, sensor data, continuous glucose data, or acceleration data indicative of physical activity.

Example 12. The computing apparatus of any of the preceding examples, wherein adjusting the one or more edge weights is further based at least in part on temporal aspects of the medical data.

Example 13. The computing apparatus of any of the preceding examples, wherein the temporal aspects comprise dates or timestamps.

Example 14. The computing apparatus of any of the preceding examples, wherein adjusting the one or more edge weights is further based at least in part on a neural network.

Example 15. The computing apparatus of any of the preceding examples, wherein adjusting the one or more edge weights is further based at least in part on mapping text and data values.

Example 16. The computing apparatus of any of the preceding examples, wherein the biological effect comprises a contraindication.

Example 17. The computing apparatus of any of the preceding examples, wherein the contraindication is one of a drug-drug contraindication or a drug-condition contraindication.

Example 18. The computing apparatus of any of the preceding examples, wherein the biological effect comprises a positive biological condition.

Example 19. The computing apparatus of any of the preceding examples, wherein a second node of interest is associated with a second biological data structure comprising a second drug identifier and a second dosage record.

Example 20. The computing apparatus of any of the preceding examples, wherein the one or more processors are further configured to perform one or more additional sequential traversals of the knowledge graph data structure until the equilibrium condition is met, by traversing from the second node of interest to a next node directly linked to the second node of interest, and iteratively traversing to respective subsequent next nodes directly linked to a previously visited node until returning to the second node of interest. The one or more processors are further configured to associate a second visit tally with each node in accordance with the one or more sequential traversals. The one or more processors are further configured to determine whether a second subset of nodes is associated with second visit tallies totaling more than a second threshold proportion of all node visits associated with the one or more additional sequential traversals, wherein the second subset of nodes represents a second biological effect associated with a second biological data structure associated with the second node of interest. The one or more processors are further configured to, based at least in part on determining that the second subset of nodes is not associated with second visit tallies totaling more than a threshold proportion of all node visits associated with the one or more additional sequential traversals, initiate transmission of a contraindication-free alert communication comprising representation of a lack of the second biological effect for the user identifier.

Example 21. The computing apparatus of any of the preceding examples, wherein the second biological data structure comprising the second drug identifier and second dosage record is selected based at least in part on minimizing a first number of visits to side-effect-associated nodes or maximizing a ratio of treatment effectiveness to treatment side effects.

Example 22. The computing apparatus of any of the preceding examples, wherein treatment effectiveness is determined by a second number of visits to nodes associated with disease amelioration.

Example 23. The computing apparatus of any of the preceding examples, treatment side effects are determined by a third number of visits to side-effect-associated nodes.

Example 24. The computing apparatus of any of the preceding examples, the contraindication alert communication comprises representation of a diagnostic test recommendation.

Example 25. The computing apparatus of any of the preceding examples, wherein the one or more processors are further configured to generate the knowledge graph data structure.

Example 26. The computing apparatus of any of the preceding examples, wherein the first biological data structure comprises one or more of a protein identifier, a biomolecule identifier, or a biological process identifier.

Example 27. A computer-implemented method, comprising: accessing a knowledge graph data structure that comprises (i) a graph-based representation associated with a user identifier, (ii) a plurality of nodes, wherein each node represents a biological data structure, (iii) a plurality of edges, wherein each of the plurality of edges (a) connects two respective nodes, (b) represents a relationship associated with the two nodes connected by the edge, (c) is associated with an edge weight, and (d) is initially associated with a respective default edge weight. The method further comprising adjusting one or more edge weights based at least in part on medical data associated with the user identifier. The method further comprising performing one or more sequential traversals of the knowledge graph data structure, starting at a node of interest, until an equilibrium condition is met when a variation in a proportion of total visits received by each of the plurality of nodes caused by the one or more sequential traversals falls below a variation threshold. The method further comprising determining whether a subset of the plurality of nodes is associated with visit tallies totaling more than a threshold associated with the proportion of total visits, wherein the subset of nodes represents a biological effect associated with the node of interest. The method further comprising based at least in part on determining that the subset of the plurality of nodes is associated with the visit tallies, initiating transmission of a contraindication alert communication.

Example 28. A non-transitory computer-readable storage medium storing instructions that, when executed by at least one processor, cause the at least one processor to access a knowledge graph data structure that comprises (i) a graph-based representation associated with a user identifier, (ii) a plurality of nodes, wherein each node represents a biological data structure, (iii) a plurality of edges, wherein each of the plurality of edges (a) connects two respective nodes, (b) represents a relationship associated with the two nodes connected by the edge, (c) is associated with an edge weight, and (d) is initially associated with a respective default edge weight. The at least one processor is further caused to adjust one or more edge weights based at least in part on medical data associated with the user identifier. The at least one processor is further caused to perform one or more sequential traversals of the knowledge graph data structure, starting at a node of interest, until an equilibrium condition is met when a variation in a proportion of total visits received by each of the plurality of nodes caused by the one or more sequential traversals falls below a variation threshold. The at least one processor is further caused to determine whether a subset of the plurality of nodes is associated with visit tallies totaling more than a threshold associated with the proportion of total visits, wherein the subset of nodes represents a biological effect associated with the node of interest. The at least one processor is further caused to, based at least in part on determining that the subset of the plurality of nodes is associated with the visit tallies, initiate transmission of a contraindication alert communication.

1. A computing apparatus comprising memory and one or more processors communicatively coupled to the memory, the one or more processors configured to:
access a knowledge graph data structure that comprises (i) a graph-based representation associated with a user identifier, (ii) a plurality of nodes, wherein each node represents a biological data structure, (iii) a plurality of edges, wherein each of the plurality of edges (a) connects two respective nodes, (b) represents a relationship associated with the two nodes connected by the edge, (c) is associated with an edge weight, and (d) is initially associated with a respective default edge weight;
adjust one or more edge weights based at least in part on medical data associated with the user identifier;
perform one or more sequential traversals of the knowledge graph data structure, starting at a node of interest, until an equilibrium condition is met when a variation in a proportion of total visits received by each of the plurality of nodes caused by the one or more sequential traversals falls below a variation threshold;
determine whether a subset of the plurality of nodes is associated with visit tallies totaling more than a threshold associated with the proportion of total visits, wherein the subset of nodes represents a biological effect associated with the node of interest; and
based at least in part on determining that the subset of the plurality of nodes is associated with the visit tallies, initiate transmission of a contraindication alert communication.

2. The computing apparatus of claim 1, wherein performing the one or more sequential traversals of the knowledge graph data structure until the equilibrium condition is met comprises:
traversing from a first node to a second node directly linked to the first node; and
iteratively traversing to subsequent nodes until returning to the first node.

3. The computing apparatus of claim 1, wherein the contraindication alert communication comprises representation of the biological effect for the user identifier.

4. The computing apparatus of claim 1, wherein the node of interest is a first node of interest associated with the first biological data structure comprising a first drug identifier and a first dosage record.

5. The computing apparatus of claim 4, wherein a second node of interest is associated with a second biological data structure comprising the first drug identifier and a second dosage record.

6. The computing apparatus of claim 5, wherein the one or more processors are further configured to:
perform one or more additional sequential traversals of the knowledge graph data structure until the equilibrium condition is met, by:
traversing from the second node of interest to a next node directly linked to the second node of interest; and
iteratively traversing to respective subsequent next nodes directly linked to a previously visited node until returning to the second node of interest;
associate a second visit tally with each node in accordance with the one or more sequential traversals;
determine whether a second subset of nodes is associated with second visit tallies totaling more than a second threshold proportion of all node visits associated with the one or more additional sequential traversals, wherein the second subset of nodes represents a second biological effect associated with a second biological data structure associated with the second node of interest; and
based at least in part on determining that the second subset of nodes is not associated with second visit tallies totaling more than a threshold proportion of all node visits associated with the one or more additional sequential traversals, initiate transmission of a contraindication alert communication comprising representation of a lack of the second biological effect for the user identifier.

7. The computing apparatus of claim 1, wherein selecting the next node to which to traverse from the first node of interest is performed randomly.

8. The computing apparatus of claim 7, wherein a first probability of selecting a node directly linked to the node of interest as the next node is proportional to a respective edge weight of the node relative to edge weights of other nodes directly linked to the node of interest.

9. The computing apparatus of claim 1, wherein selecting the subsequent next node to which to traverse from the previously visited node is performed randomly.

10. The computing apparatus of claim 9, wherein a second probability of selecting a node directly linked to the previously visited node is proportional to a respective edge weight of the node relative to edge weights of other nodes directly linked to the previously visited node.

11. The computing apparatus of claim 1, wherein the medical data associated with the user identifier comprises one or more of clinical notes data, comorbidities data, current medications data, lab values data, insurance claims data, demographic data, medical images data, sensor data, continuous glucose data, or acceleration data indicative of physical activity.

12. The computing apparatus of claim 1, wherein adjusting the one or more edge weights is further based at least in part on temporal aspects of the medical data.

13. The computing apparatus of claim 12, wherein the temporal aspects comprise dates or timestamps.

14. The computing apparatus of claim 1, wherein adjusting the one or more edge weights is further based at least in part on a neural network.

15. The computing apparatus of claim 1, wherein adjusting the one or more edge weights is further based at least in part on mapping text and data values.

16. The computing apparatus of claim 1, wherein the biological effect comprises a contraindication.

17. The computing apparatus of claim 16, wherein the contraindication is one of a drug-drug contraindication or a drug-condition contraindication.

18. The computing apparatus of claim 1, wherein the biological effect comprises a positive biological condition.

19. The computing apparatus of claim 4, wherein a second node of interest is associated with a second biological data structure comprising a second drug identifier and a second dosage record.

20. The computing apparatus of claim 19, wherein the one or more processors are further configured to:
perform one or more additional sequential traversals of the knowledge graph data structure until the equilibrium condition is met, by:
traversing from the second node of interest to a next node directly linked to the second node of interest; and
iteratively traversing to respective subsequent next nodes directly linked to a previously visited node until returning to the second node of interest;
associate a second visit tally with each node in accordance with the one or more sequential traversals;
determine whether a second subset of nodes is associated with second visit tallies totaling more than a second threshold proportion of all node visits associated with the one or more additional sequential traversals, wherein the second subset of nodes represents a second biological effect associated with a second biological data structure associated with the second node of interest; and
based at least in part on determining that the second subset of nodes is not associated with second visit tallies totaling more than a threshold proportion of all node visits associated with the one or more additional sequential traversals, initiate transmission of a contraindication-free alert communication comprising representation of a lack of the second biological effect for the user identifier.

21. The computing apparatus of claim 20, wherein the second biological data structure comprising the second drug identifier and second dosage record is selected based at least in part on minimizing a first number of visits to side-effect-associated nodes or maximizing a ratio of treatment effectiveness to treatment side effects.

22. The computing apparatus of claim 21, wherein treatment effectiveness is determined by a second number of visits to nodes associated with disease amelioration.

23. The computing apparatus of claim 22, wherein treatment side effects are determined by a third number of visits to side-effect-associated nodes.

24. The computing apparatus of claim 2, wherein the contraindication alert communication comprises representation of a diagnostic test recommendation.

25. The computing apparatus of claim 1, wherein the one or more processors are further configured to:
generate the knowledge graph data structure.

26. The computing apparatus of claim 3, wherein the first biological data structure comprises one or more of a protein identifier, a biomolecule identifier, or a biological process identifier.

27. A computer-implemented method, comprising:
accessing a knowledge graph data structure that comprises (i) a graph-based representation associated with a user identifier, (ii) a plurality of nodes, wherein each node represents a biological data structure, (iii) a plurality of edges, wherein each of the plurality of edges (a) connects two respective nodes, (b) represents a relationship associated with the two nodes connected by the edge, (c) is associated with an edge weight, and (d) is initially associated with a respective default edge weight;
adjusting one or more edge weights based at least in part on medical data associated with the user identifier;
performing one or more sequential traversals of the knowledge graph data structure, starting at a node of interest, until an equilibrium condition is met when a variation in a proportion of total visits received by each of the plurality of nodes caused by the one or more sequential traversals falls below a variation threshold;
determining whether a subset of the plurality of nodes is associated with visit tallies totaling more than a threshold associated with the proportion of total visits, wherein the subset of nodes represents a biological effect associated with the node of interest; and
based at least in part on determining that the subset of the plurality of nodes is associated with the visit tallies, initiating transmission of a contraindication alert communication.

28. A non-transitory computer-readable storage medium storing instructions that, when executed by at least one processor, cause the at least one processor to:
access a knowledge graph data structure that comprises (i) a graph-based representation associated with a user identifier, (ii) a plurality of nodes, wherein each node represents a biological data structure, (iii) a plurality of edges, wherein each of the plurality of edges (a) connects two respective nodes, (b) represents a relationship associated with the two nodes connected by the edge, (c) is associated with an edge weight, and (d) is initially associated with a respective default edge weight;
adjust one or more edge weights based at least in part on medical data associated with the user identifier;
perform one or more sequential traversals of the knowledge graph data structure, starting at a node of interest, until an equilibrium condition is met when a variation in a proportion of total visits received by each of the plurality of nodes caused by the one or more sequential traversals falls below a variation threshold;
determine whether a subset of the plurality of nodes is associated with visit tallies totaling more than a threshold associated with the proportion of total visits, wherein the subset of nodes represents a biological effect associated with the node of interest; and
based at least in part on determining that the subset of the plurality of nodes is associated with the visit tallies, initiate transmission of a contraindication alert communication.
